(19) United States
(12) Patent Application Publication
(10) Pub. No.: US 2026/0008985 A1
(43) Pub. Date: Jan. 8, 2026

(54) CELL OPERATION DEVICE FOR CELL SPHEROIDS CULTURE AND ANALYSIS

(71) Applicant: National Health Research Institutes, Miaoli County (TW)

(72) Inventors: Chia-Hsien HSU, Miaoli County (TW); Chuan-Feng YEH, Miaoli County (TW); Chi-Han LI, Miaoli County (TW); Jing-Yi YANG, Westfield, IN (US)

(21) Appl. No.: 19/258,270

(22) Filed: Jul. 2, 2025

Related U.S. Application Data
(60) Provisional application No. 63/667,288, filed on Jul. 3, 2024.

(51) Int. Cl. C12M 3/06; C12M 1/00; C12M 1/12; C12M 1/32

(52) U.S. Cl. CPC C12M 23/16; C12M 23/12; C12M 23/22; C12M 25/01

(57) ABSTRACT

This disclosure relates to a cell operation device for the generation, culture, and collection of cellular spheroids. The device comprises a plurality of independently operable units, each including a vertically aligned first chamber and second chamber. The first chamber is configured with a microchannel and an array of through holes that enable the formation of uniform hanging drops, facilitating spheroid formation under controlled conditions. The second chamber is positioned below to receive and immobilize the spheroids for imaging or downstream processing. This modular design enables scalable and reproducible 3D cell culture suitable for biomedical research and drug screening applications.

### CROSS REFERENCE TO RELATED APPLICATIONS

This application claims priority of U.S. Provisional Application No. 63/667,288 filed on Jul. 2, 2024 under 35 U.S.C. § 119(e), the entire contents of which are hereby incorporated by reference

### BACKGROUND OF THE INVENTION

### Technical Field of the Invention

The present invention relates generally to device and methods for cell operation, and more particularly to device and methods for efficiently handling and analyzing 3D cell spheroids with high integrity and throughput.

### Background

3D cell spheroid models are increasingly being adopted in basic research, drug screening, and precision medicine applications due to their physiological relevance compared to traditional 2D cultures. However, the analysis of spheroid characteristics, particularly via high-content imaging, remains labor-intensive and inefficient. Standard processes such as transfer, centrifugation, and liquid exchange can severely compromise the structural integrity of spheroids, leading to cell damage and loss. These factors significantly reduce the throughput of spheroid-based assays and limit their utility for large-scale applications.

Conventional procedures for handling spheroids typically involve manual manipulation using micropipettes, transfer to centrifuge tubes for washing steps, fixation, permeabilization, immunostaining, and subsequent relocation to imaging-compatible platforms such as glass-bottom dishes. These multistep processes are not only time-consuming but also increase the risk of contamination and physical damage to the spheroids. Moreover, uniform spheroid generation is often carried out using low-attachment, round-bottom well plates, which, while somewhat effective for culture, remain inefficient for downstream processing and imaging workflows.

Advancements have been made to simplify spheroid culture and handling. For instance, the hanging drop well plate system developed by Insphero enables spheroid formation in suspended droplets. Although this method improves initial formation, the transfer process—where liquid is added to collapse the droplet and allow the spheroid to settle into a lower well—poses challenges. During immunostaining or washing steps, micropipette-based liquid exchange within these wells often results in direct contact with the spheroid, increasing the likelihood of cell loss or damage. Furthermore, the plastic bottom of these wells compromises optical clarity, making them suboptimal for high-resolution imaging.

Another method, described in recent literature (Kim, H., Roh, H., Kim, H., & Park, J. K. (2021)), uses a droplet-on-micropillar approach to facilitate spheroid transfer. This system consists of a PDMS-based droplet array chip (DAC) aligned with a pillar array chip (PAC) to achieve arrayed transfer of spheroids. However, this approach is limited by the complexity of spheroid culture in suspended droplets and the requirement for precise DAC-PAC alignment during transfer. The bulky device architecture further hinders high-resolution imaging, thus limiting its effectiveness in downstream applications.

Therefore, the present invention provides a novel cell operation device that overcomes the limitations of existing methods, enabling efficient, high-throughput, and gentle handling of 3D cell spheroids.

To date, no commercial system has successfully addressed all of the challenges associated with high-throughput, high-integrity spheroid operations—namely, gentle handling, precise liquid exchange, minimized loss, and compatibility with high-content imaging. Therefore, there exists a critical need for a device and method that enables efficient, scalable, and minimally invasive manipulation of 3D cell spheroids across various stages of biological analysis.

The present invention further provides a cell operation method that ensures minimal cell damage and loss while maintaining spheroid integrity for downstream analysis.

### SUMMARY OF INVENTION

In one aspect, the present invention provides a cell operation device, comprising a plurality of independently operable chip units. Each chip unit comprises a first chamber positioned above a second chamber, arranged vertically. The first chamber comprises a microchannel and an array of through-holes disposed on the bottom surface of the microchannel, which are configured to facilitate droplet formation. The second chamber is configured to receive a plurality of spheroids from the formed droplets.

In some embodiments, each first chamber further includes a first injection port in fluid communication with the microchannel, adapted to receive a sample solution such as a cell suspension.

In some embodiments, each first chamber further comprises a reservoir in fluid communication with the microchannel, which is designed to generate a hydrostatic pressure difference that enables droplet formation.

In some embodiments, the second chamber is disposed on a bottom substrate comprising a transparent glass layer, allowing for optical observation.

In some embodiments, the glass layer has a thickness ranging from approximately 0.1 mm to 0.17 mm, providing structural support while maintaining imaging compatibility.

In some embodiments, the first chamber comprises a second injection port, fluidly connected to the second chamber, allowing infusion of medium into the second chamber until it contacts the hanging droplets.

In some embodiments, each chip unit further comprises:
(a) an inlet formed on the first chamber, configured to introduce medium and fluidly connected to the second chamber via internal channels; and
(b) an outlet also formed on the first chamber, configured to remove medium and fluidly connected via internal channels to the second chamber.

In some embodiments, the first chamber includes a plurality of vertically extending pillars directed toward the second chamber, which enhance mechanical connectivity and structural stability of the device.

In some embodiments, the area of the microchannel ranges from approximately 1×1 mm² to 86×128 mm², and the height of the microchannel ranges from approximately 0.1 mm to 5 mm.

In some embodiments, each through-hole in the array has a diameter ranging from 0.05 mm to 5 mm, and a depth ranging from 0.1 mm to 5 mm.

In some embodiments, the second chamber comprises a plurality of collection wells disposed on the transparent glass layer. Each collection well is positioned directly beneath a corresponding droplet formed at the through-holes.

In some embodiments, each collection well is shaped as a geometric prism, selected from the group consisting of: rectangular, pentagonal, heptagonal, octagonal, triangular, square, cylindrical, or trapezoidal prism.

In some embodiments, each collection well has a planar area ranging from approximately 0.05×0.05 mm² to 86×128 mm², and a height ranging from 0.05 mm to 5 mm.

In some embodiments, the first and second chambers are made of one or more materials selected from the group consisting of: polydimethylsiloxane (PDMS), polycarbonate (PC), polymethyl methacrylate (PMMA), polyolefin copolymer (POC), polystyrene (PS), polypropylene (PP), glass, and hydrogel.

In another aspect, the present invention provides a method for operating the above-described device, comprising:
(a) loading a cell suspension into the first chamber through the first injection port;
(b) introducing a culture medium into the reservoir to generate hydrostatic pressure, thereby forming hanging droplets at the array of through-holes;
(c) introducing medium into the second chamber via the second injection port until it contacts the hanging droplets.

In some embodiments of the method, operation further includes:
(d) performing medium replacement by removing and adding fresh medium at a volume ranging from 1× to 3×the total volume of the microchannel; and
(e) placing the assembled device on a microscope stage to conduct high-resolution confocal imaging of the collected spheroids.

### BRIEF DESCRIPTION OF THE DRAWINGS

FIG. 1 is the illustration of the device design.

FIG. 2 depicts an example of the device

FIG. 3 illustrates an exploded view of the device.

FIG. 4 is the cross-sectional view of the first chamber loading with sample solution.

FIG. 5 is the cross-sectional view of the second chamber loading with sample solution.

FIG. 6 is the top view of the device.

FIG. 7 is the potential second chamber design.

FIG. 8 is the potential collection well designs.

FIG. 9 presents another possible design of the second chamber design.

FIG. 10 is the top view of the potential second chamber design.

FIG. 11 is the side view of the potential second chamber design.

FIG. 12 illustrates another possible design showing the side view of the second chamber design.

FIGS. 13A-13H show the spheroid formation with ULA cultures and device cultures.

FIGS. 14A-14D show the validation of spheroid collection efficiency with the device

### DETAILED DESCRIPTION OF THE INVENTION

Other features and advantages of the present invention will be further exemplified and described in the following examples, which are intended to be illustrative only and not to limit the scope of the invention.

The embodiments of the invention will be apparent from the following detailed description, which proceeds with reference to the accompanying drawings, wherein the same references relate to the same elements.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments.

As used herein, “have”, “has”, “having”, “include”, “includes”, “including”, “comprise”, “comprises”, “comping” or the like are used in their open ended inclusive sense, and generally mean “include, but not limited to”, “includes, but not limited to”, or “including, but not limited to”. “Optional” or “optionally” means that the subsequently described event, circumstance, or component, can or cannot occur, and that the description includes instances where the event, circumstance, or component, occurs and instances where it does not. Unless otherwise specified, “a,” “an,” “the,” and “at least one” are used interchangeably and mean one or more than one.

Also herein, the recitations of numerical ranges by endpoints include all numbers subsumed within that range (e.g., I to 5 includes 1. 1.5, 2, 2.75, 3.80, 4, 5, etc.). Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

### Example 1. Device Design, and Assembly

### Device Fabrication

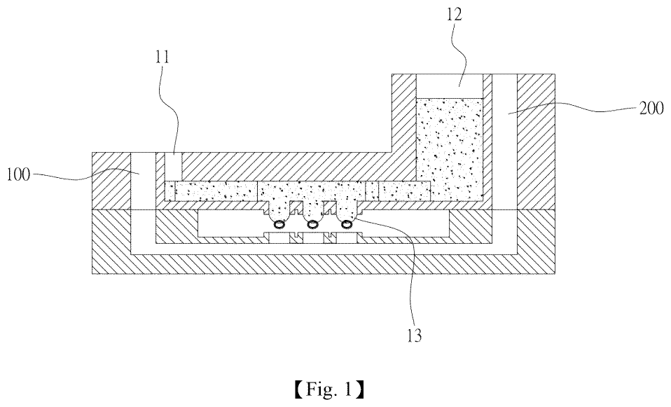

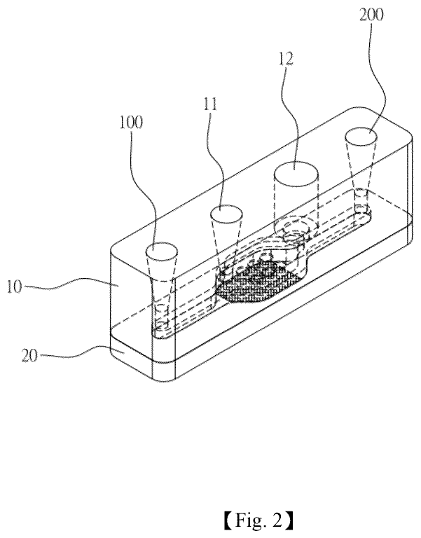

As shown in FIGS. 1 and 2, the disclosed device offers significant advantages, including, for example: enabling the formation, culture, and collection of uniform 3D spheroids within a single integrated system, thereby eliminating the need for transferring spheroids between separate vessels during drug screening, staining, or imaging. This configuration minimizes handling-related spheroid loss or disruption and allows direct optical access to spheroids through a transparent bottom surface, facilitating downstream analysis without additional transfer steps. In embodiments, the device comprises at least a first chamber (10) and a second chamber (20) arranged in a vertically stacked configuration, and assembled into a unified structure.

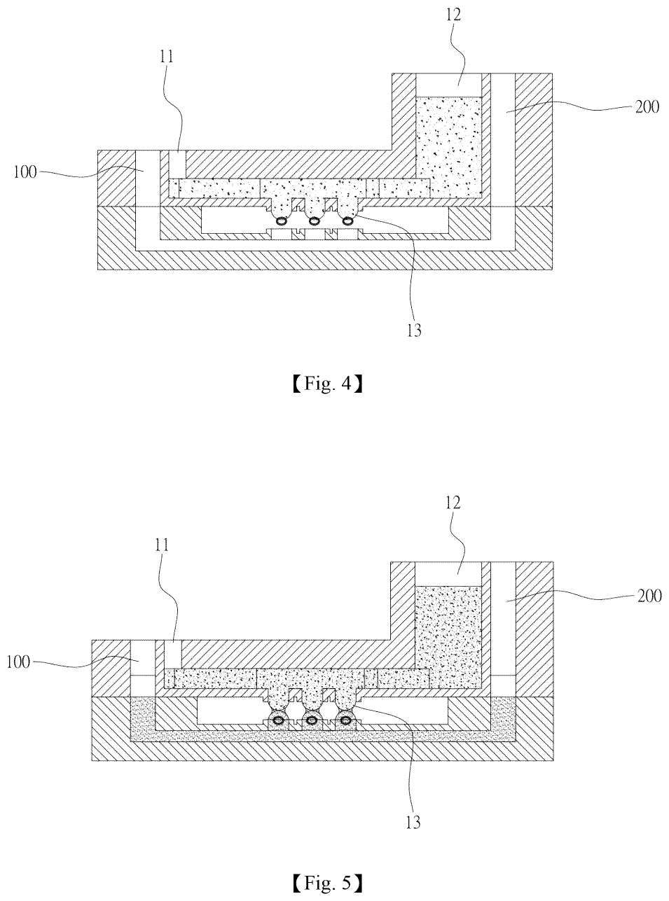

In some embodiments, the first chamber is positioned vertically above the second chamber, such that the array of through-holes (13) of the first chamber is aligned with the collection wells (21) of the second chamber. This vertical arrangement facilitates gravitational transfer of spheroids from the an array of through-holes (13) to the collection wells (21), thereby simplifying the transition from spheroid formation to analysis (FIG. 4-5).

The first chamber (10) is configured to accommodate the cell loading and droplet formation process. It includes at least one injection port (11) for introducing a cell suspension into the microchannel region.

In some embodiments, the first chamber (10) comprises an elongated microchannel, which serves as the main passage for cell suspension and medium flow. The microchannel can have, for example, a planar area ranging from about 3×15 mm² to about 6×25 mm², more preferably from about 4×17 mm² to about 6×22 mm², and most preferably from about 5×18 mm² to about 5×20 mm², including intermediate values and ranges, depending on factors such as desired droplet number, cell density, and format compatibility. The channel height can be, for example, from about 0.8 mm to about 1.5 mm, more preferably from about 1.0 mm to about 1.3 mm, and most preferably around 1.2 mm, again depending on fluid volume and droplet stability requirements.

The bottom surface of the microchannel in the first chamber (10) is structured with an array of through-holes (13), which allow the formation of hanging droplets via hydrostatic pressure (FIG. 1, 3). These through-holes are dimensioned to promote controlled droplet detachment and hanging drop stability, enabling gradual 3D spheroid formation through gravitational sedimentation of cells. In embodiments, the diameter of each droplet formation hole can be, for example, from about 0.5 mm to about 1.5 mm, more preferably from about 0.8 mm to about 1.3 mm, and most preferably from about 1.0 mm to about 1.2 mm. The depth of each hole can range, for example, from about 0.3 mm to about 1.0 mm, more preferably from about 0.5 mm to about 0.9 mm, and most preferably from about 0.7 mm to about 0.8 mm, including intermediate values and ranges, depending on fluid viscosity, cell type, and desired droplet volume.

These through-holes are further designed to maintain droplet stability, prevent backflow or leakage, and ensure gravitational collection of matured spheroids into corresponding locations in the lower chamber. The hole configuration thereby supports high-throughput, reproducible spheroid generation across a large number of sites.

A reservoir (12) is connected to the first chamber to supply fresh culture medium (FIG. 1). The reservoir is positioned at a higher elevation relative to the first injection port of the first chamber, thereby generating a hydrostatic pressure gradient that facilitates fluid flow. Additionally, the reservoir has a larger volume compared to the first chamber, ensuring a continuous and sustained supply of culture medium over time. During operation, the hydrostatic pressure drives the cell-containing liquid through the array of droplet formation holes located at the bottom surface of the microchannel, initiating droplet creation. This pressure-driven flow not only enables consistent droplet generation but also supports continuous nutrient delivery to the developing spheroids in the second chamber.

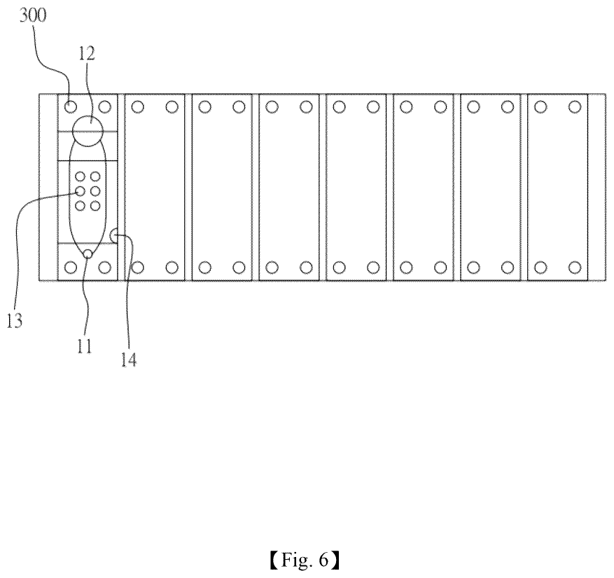

The first chamber further comprises a second injection port (14) fluidically connected to the second chamber via an internal flow path (FIG. 6). This configuration allows introduction of culture medium or reagents into the second chamber, facilitating detachment of spheroids from the hanging droplets above and their subsequent collection within the wells of the second chamber. The fluid flow generated through this port enables efficient spheroid transfer while minimizing mechanical disruption.

In some embodiments, the device is designed to enable stable vertical stacking, precise chamber alignment, and non-disruptive modular disassembly, thereby supporting an efficient transition between spheroid culture and subsequent imaging or analytical procedures. To achieve this, the device includes a set of modular alignment structures, which, in one example, comprise four copper pillars (300). These pillars are spatially arranged to align the first chamber above the second chamber with high precision. The copper pillars provide structural support for stable assembly and allow the two chambers to be easily separated post-culture, minimizing handling-induced disruption to the spheroids retained in the lower chamber.

The second chamber (20) is located directly beneath the array of through-holes and comprises a transparent glass substrate patterned with a plurality of collection wells (21) (FIG. 2). These wells are positioned to receive the spheroids released from the droplets. The transparency of the base allows for direct imaging of the spheroids, including under confocal or fluorescence microscopy.

Each collection well may have, for example, a planar area ranging from about 1.0×6 mm² to about 2.0×12 mm², more preferably from about 1.2×8 mm² to about 1.8×10 mm², and most preferably around 1.6×9 mm², with a depth ranging from about 0.8 mm to about 1.5 mm, more preferably from about 1.0 mm to about 1.3 mm, and most preferably about 1.2 mm, including intermediate values and ranges depending on the target spheroid size and fluid exchange needs. The total device footprint can be, for example, from about 20×60 mm² to about 30×80 mm², more preferably from about 22×70 mm² to about 27×75 mm², and most preferably around 25×75 mm², which is compatible with standard microscope stages. This dimensional configuration allows direct in situ observation of spheroids through the transparent substrate without requiring transfer, thereby preserving spatial arrangement and minimizing sample loss. Collected spheroids may undergo staining, imaging, or biochemical assays directly within the wells, supporting streamlined and non-disruptive analysis workflows.

In some embodiments, the transparent glass substrate forming the base of the second chamber has a thickness ranging from about 0.1 mm to about 0.17 mm. This thickness range is selected to balance optical transparency suitable for high-resolution imaging with sufficient mechanical strength to maintain structural integrity during device handling and operation.

The collection wells patterned on the transparent glass substrate of the second chamber may have a variety of geometric shapes to optimize spheroid confinement and culture conditions. Exemplary well shapes include rectangles, pentagons, heptagons, octagons, triangles, squares, cylindrical posts, trapezoidal posts, or other polygonal geometries. Such diversity in well shape allows customization of the device for specific cell types, spheroid sizes, or imaging modalities.

The first and second chambers of the device can be fabricated from various materials, including but not limited to polydimethylsiloxane (PDMS), polycarbonate (PC), polymethyl methacrylate (PMMA), polyolefin copolymers (POC), polystyrene (PS), polypropylene (PP), glass, and hydrogel materials. Selection of the chamber materials can be based on desired optical properties, biocompatibility, fabrication methods, and mechanical requirements.

In some embodiments, the devices are configured such that cells cultured in the devices form spheroids. For example, the wells in which cells are grown can be non-adherent to cells to cause the cells in the wells to associate with each other and form spheres. The spheroids expand to size limits imposed by the geometry of the wells. In some embodiments, the wells are coated with an ultra-low binding material to make the wells non-adherent to cells.

A wide variety of cell types may be cultured. In some embodiments, a spheroid contains a single cell type. In some embodiments, a spheroid contains more than one cell type. In some embodiments, where more than one spheroid is grown, each spheroid is of the same type, while in other embodiments, two or more different types of spheroids are grown. Cells grown in spheroids may be natural cells or altered cells. e.g, cell comprising one or more non-natural genetic alterations). In some embodiments, the cell is a somatic cell. In some embodiments, the cell is a stem cell or progenitor cell (e.g, embryonic stem cell, induced pluripotent stem cell) in any desired state of differentiation (e.g., pluripotent, multi-potent, fate determined, immortalized, etc.). In some embodiments, the cell is a discase cell or discase model cell. For example, in some embodiments, the spheroid comprises one or more types of cancer cells or cells that can be induced into a hyper-proliferative state (e.g., transformed cells). Cells may be from or derived from any desired tissue or organ type, including but not limited to, adrenal, bladder, blood vessel, bone, bone marrow, brain, cartilage, cervical, corneal, endometrial, esophageal, gastrointestinal, immune system (e.g, T lymphocytes, B lymphocytes, leukocytes, macrophages, and dendritic cells), liver, lung, lymphatic, muscle (e.g., cardiac muscle), neural, ovan, pancreatic (e.g., islet cells), pitary, prostate, renal, salivary, skin, tendon, testicular, and thyroid. In some embodiments, the cells are mammalian cells (e.g., human, mice, rat, rabbit, dog, cat, cow, pig, chicken, goat, horse, etc.).

### Example 2. Device Operation

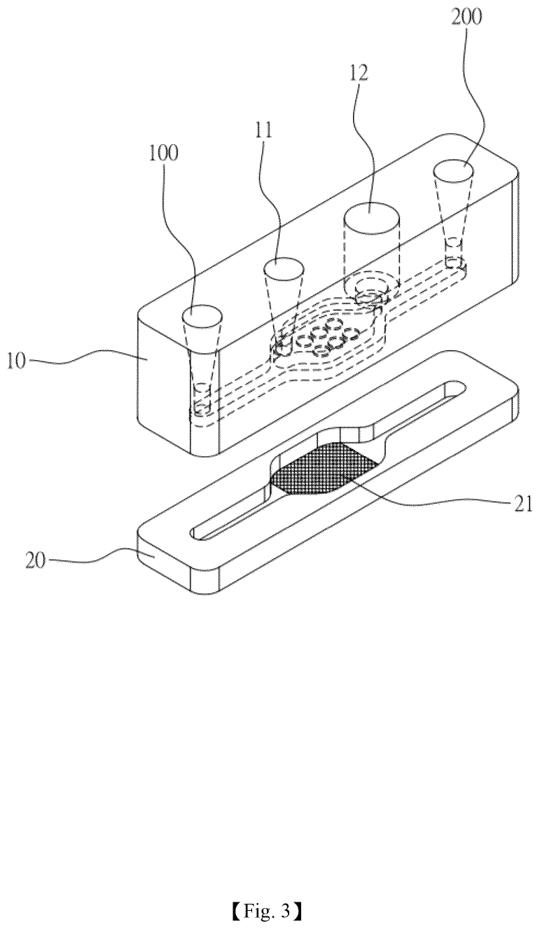

As shown in FIG. 3, a cell suspension is loaded into the microchannel of the first chamber through the injection port (11). Then, culture medium is introduced into the reservoir (12) to generate a hydrostatic pressure difference across the array of through-holes (13). This process results in the formation of hanging droplets at the holes, and the cells within the droplets sediment under gravity, gradually aggregating to form uniform 3D spheroids.

To collect spheroids for imaging or downstream analysis, medium is introduced through the second injection port (14) of the device (FIG. 4-5). When the rising fluid front contacts the droplets, the spheroids detach and fall into the corresponding collection wells (21). The wells are geometrically confined to retain the spheroids during further liquid exchanges (e.g., during staining). This design allows liquid handling and reagent exchange to occur without spheroid displacement or mechanical disruption.

Following spheroid collection, the entire device can be directly mounted onto a microscope stage. This enables high-resolution confocal imaging without the need to transfer spheroids to another container, minimizing sample loss and preserving spatial integrity.

### Example 3. Potential Designs of Confinement Structures in the Chip

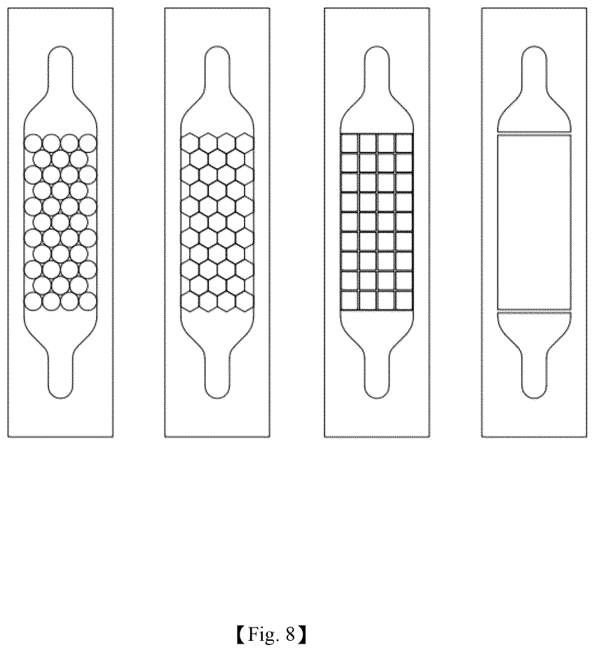

To further enhance the spheroid stability during liquid exchange, different confinement structures were incorporated into the microfluidic chip to maintain spheroid positioning. As illustrated in FIG. 8, four exemplary confinement designs are proposed.

In some embodiments of the invention, the confinement region includes an array of circular wells with diameters ranging from 0.8 to 1.5 mm, and depths ranging from 0.5 to 1 mm. These wells are evenly distributed to physically restrict each spheroid in place.

In some embodiments of the invention, hexagonal wells with side lengths ranging from 0.5 to 1 mm and depths from 0.5 to 1 mm are arranged to achieve a close-packed configuration, maximizing spatial utilization and minimizing fluid shear stress.

In some embodiments of the invention, square wells with side lengths ranging from 0.6 to 1.2 mm and depths from 0.5 to 1 mm, forming a regular grid layout to prevent spheroid displacement during flow-based liquid exchange.

In some embodiments of the invention, uses a mesh-type restriction layer without individually separated wells, featuring a central cavity with a width ranging from 3 to 6 mm and a depth of 1 mm, offering bulk confinement of multiple spheroids.

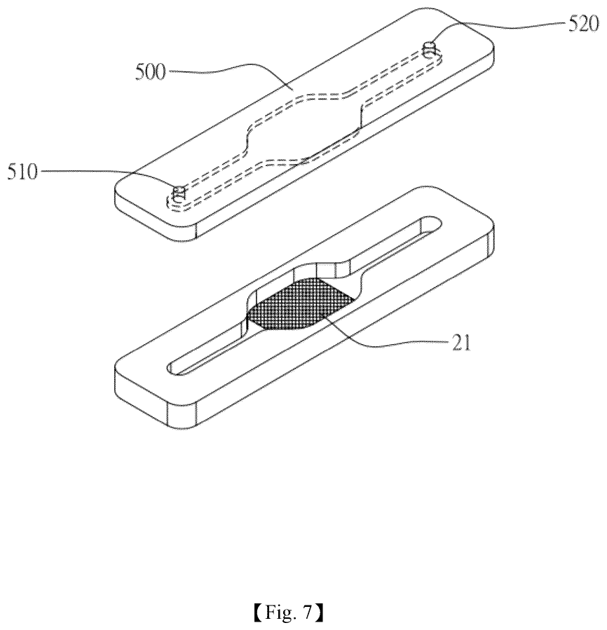

In operation, the device enables various modes of liquid exchange without removing the collection wells containing spheroids (FIG. 7).

In some embodiments of the invention, the chip operates in an enclosed channel format, where liquid is exchanged directly above the confinement wells through a continuous microfluidic channel while maintaining spheroid integrity.

In some embodiments of the invention, after removing the hanging droplet chip, a top cover (500) is added to form a sealed flow channel for liquid replacement (FIG. 7).

In some embodiments of the invention, liquid exchange is conducted via an open-slot trench after removing the hanging droplet chip, allowing easy manual pipetting while keeping spheroids immobilized within the confinement wells.

These confinement designs enable flexible and robust medium exchange, ensuring cell viability and spheroid integrity during drug testing or immunostaining procedures. Furthermore, the device can be directly transferred for high-resolution imaging and analysis without additional handling steps.

### Example 4. Alternative Second Chamber Configuration with Integrated Collection Cavity

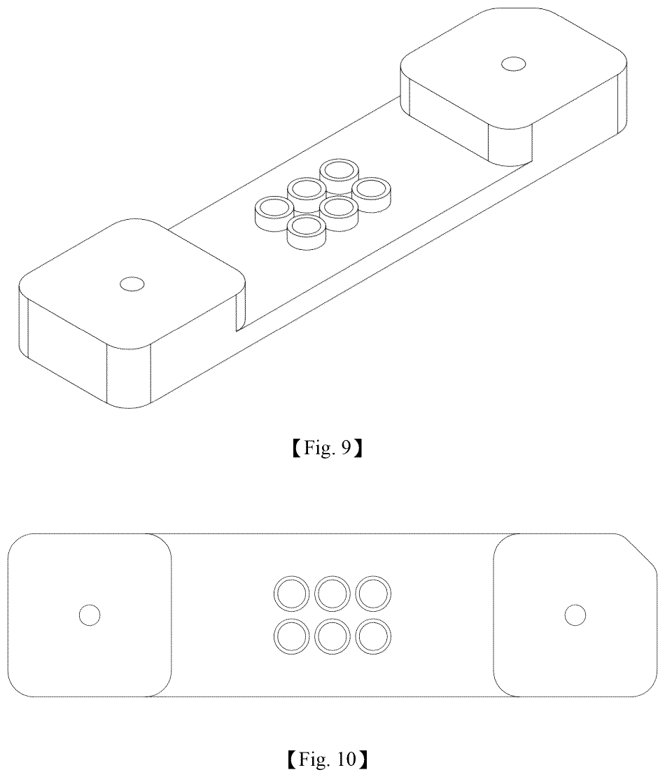

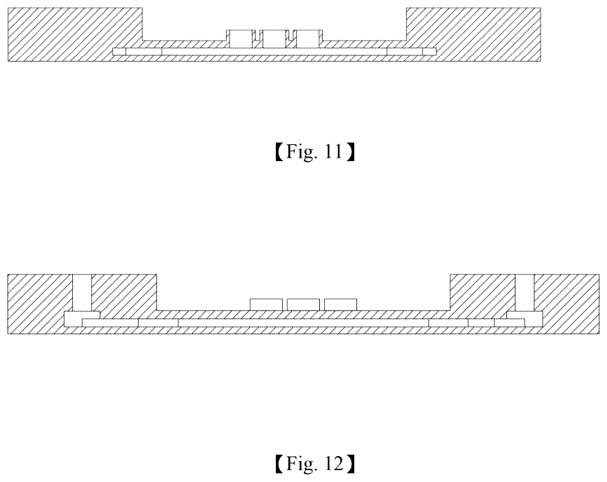

FIGS. 9 through 12 illustrate an alternative embodiment of the second chamber featuring an integrated concave collection cavity formed beneath the array of through-holes. In this configuration, the second chamber comprises a recessed basin-like structure directly aligned with the array of through-holes in the first chamber. The cavity has a depth and curvature designed to accommodate both rising fluid and sedimented spheroids, ensuring consistent positioning of collected samples.

In some embodiments, the device incorporates a confinement channel structure beneath the hanging drop chip that enables spheroid collection and liquid exchange without requiring removal of the droplet-forming unit. As shown in the cross-sectional and top views of the device (FIG. 9-10), the collection wells is formed with a concave channel geometry aligned directly beneath the through-holes of the first chamber.

In some embodiments, the second chamber further comprises one or more fluid inlets and outlets configured for controlled addition and removal of liquid medium. These ports may be positioned on lateral or basal surfaces of the second chamber, and are fluidly connected to the interior collection cavity or wells. Through these access points, culture medium or reagents can be introduced or exchanged without disrupting the spheroids residing in the chamber.

During operation, the hanging drop chip remains assembled atop the second chamber. A liquid is introduced into the second chamber through a side inlet, causing the fluid level within the cavity to gradually rise. As the fluid surface approaches the array of through holes, it forms a convex meniscus. Once contact is made between the fluid and the hanging droplets, the droplets merge with the rising fluid, triggering release of the encapsulated spheroids. These spheroids are then collected via gravitational sedimentation into the concave cavity located at the bottom of the second chamber.

This design enables medium replacement and reagent delivery to occur in a non-invasive manner, minimizing hydrodynamic shear and preventing accidental displacement or loss of spheroids during fluid exchange. By maintaining the integrity and localization of the cultured spheroids throughout the medium replacement process, the device supports extended culture durations, repeated staining or washing steps, and high-resolution imaging workflows—all within a closed, stable environment.

### Example 5. Uniformity of Single Spheroid Generation Using the Device

The uniformity of spheroid formation is a critical factor for ensuring reproducibility and precision in drug testing. Variations in the size and shape of spheroids can affect the tumor microenvironment, influenced by non-cellular factors such as nutrient distribution, pH gradients, and oxygen gradients. Therefore, maintaining the uniformity of spheroids is essential for obtaining reliable and reproducible results in cancer research and drug testing.

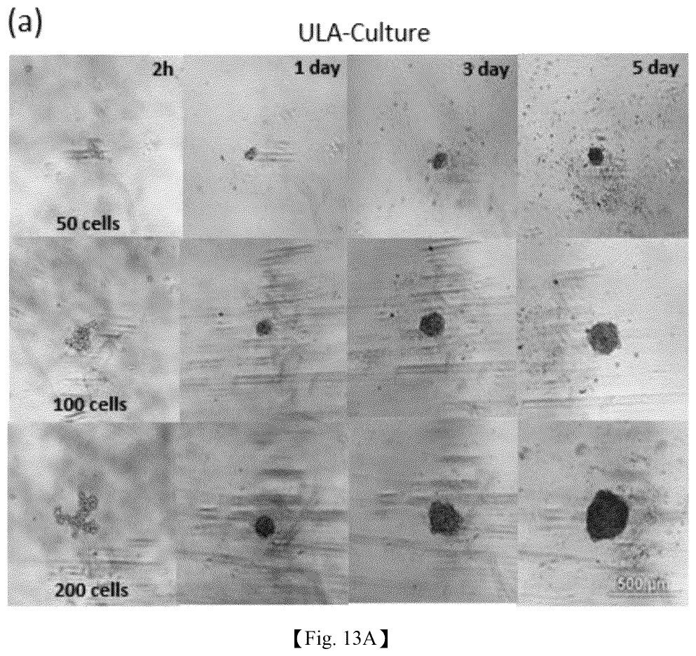

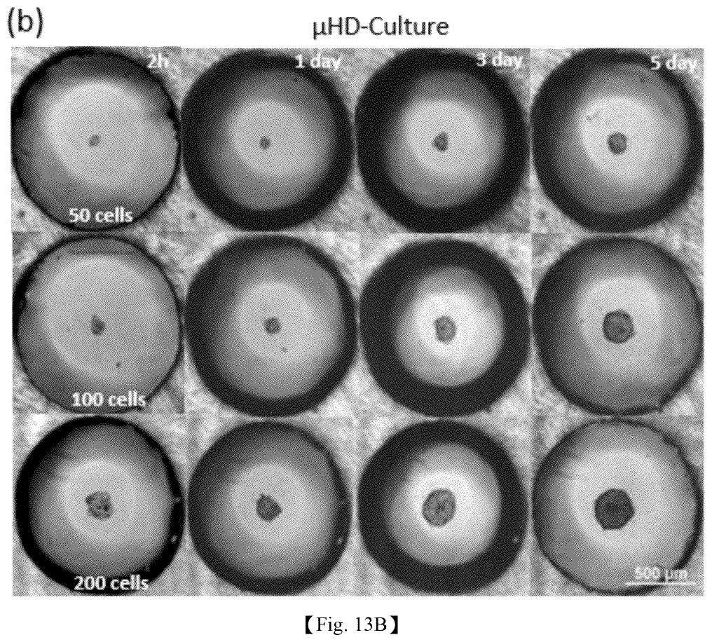

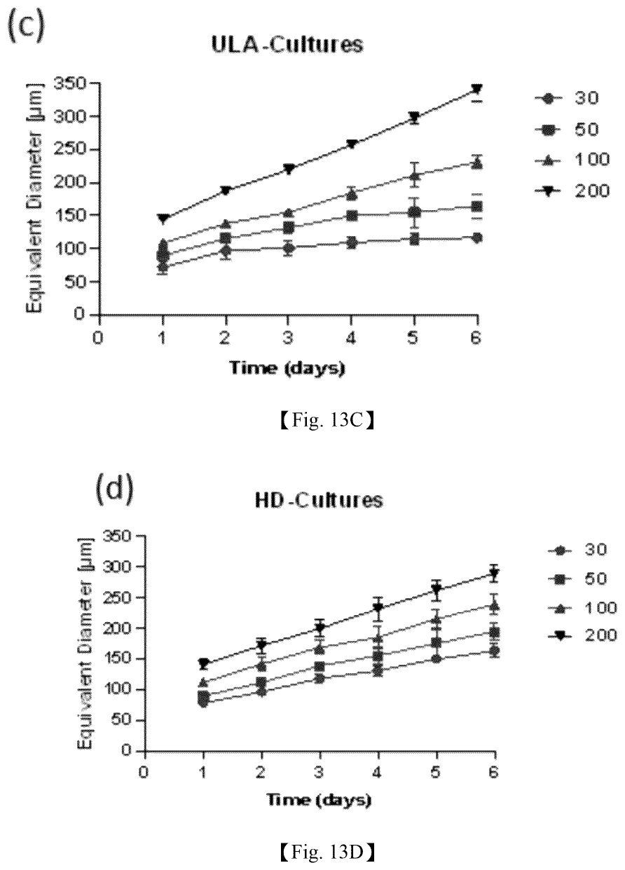

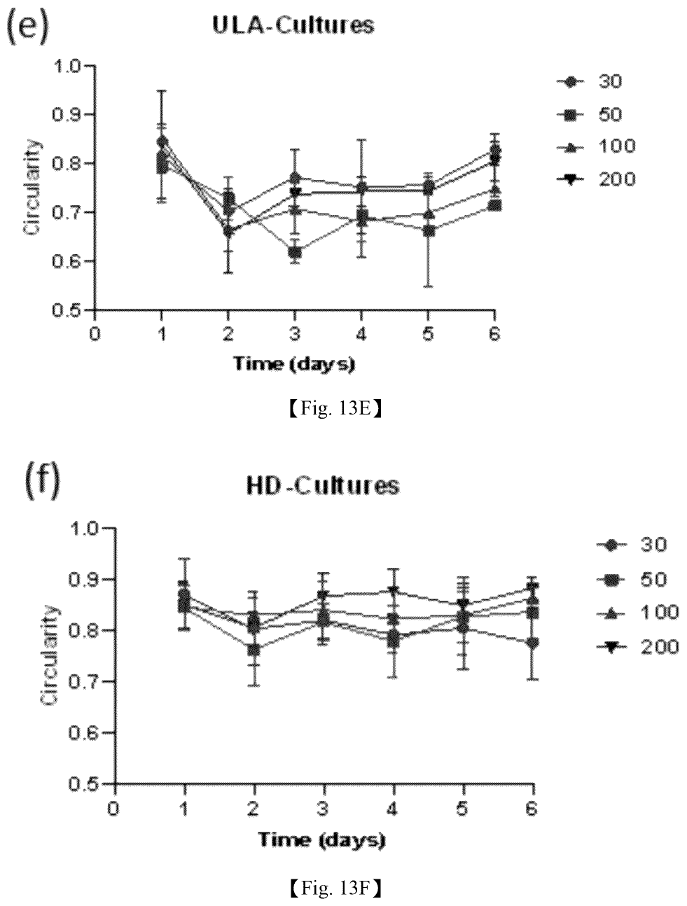

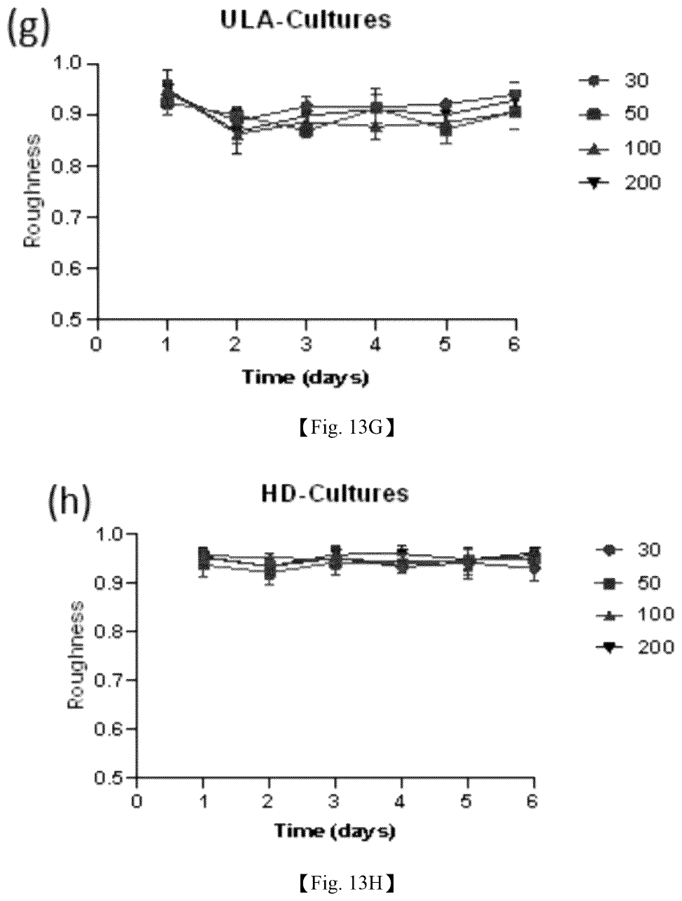

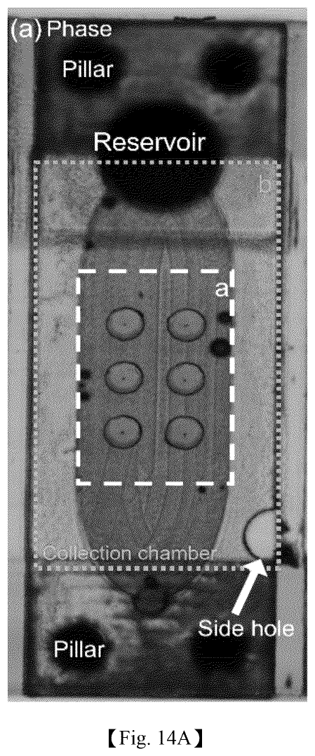

A standard method widely used for single 3D spheroid formation is the Ultra-Low Attachment (ULA) plate. In this example, the ULA plate is used as a comparison tool to evaluate the spheroid characteristics formed using the device described herein. As shown in FIG. 13a, U87 cancer cells cultured in U-shape multi-well plates with ultra-low attachment form 3D cancer spheroids from different initial cell numbers over time. In contrast, FIG. 13b illustrates the culture using the device described in this invention.

After 2 hours of cell seeding, cells cultured on the device (FIG. 13b) form nearly complete spheroid aggregations. However, as shown in FIG. 13a, cells cultured on ULA plates are still in the process of aggregation and have not yet reached complete aggregation after 2 hours. These observations suggest that cell aggregation efficiency is significantly better in the device compared to the ULA plates. The enhanced aggregation is likely due to the smaller diameter of the μ-HD wells (1,000 μm) used in the device, which results in greater droplet curvature. In contrast, the ULA plates have larger diameters (10,000 μm), leading to less curvature. This increased droplet curvature on the HD chips promotes rapid aggregation within 2 hours due to the influence of gravity.

The quantitative results shown in FIG. 9c demonstrate that spheroid diameters, both in ULA plates and the device, increase over time, indicating continuous cell division and growth. Furthermore, the initial sizes of spheroids can be controlled by varying the initial cell numbers, ensuring consistent and reproducible results. These data indicate that the cells maintain their physiological properties under the optimal culture conditions provided by the device, allowing for continuous expansion.

Interestingly, the spheroids cultured in the device exhibit an average circularity of 0.75, higher than the 0.65 observed for spheroids cultured in ULA plates. Additionally, the average roughness of device-cultured spheroids is 0.9, compared to 0.85 for spheroids cultured in ULA plates. These results indicate that the device enables the generation of uniform spheroids in both size and shape, making it suitable for use in drug screening applications.

### Example 6: Validation of Spheroid Collection Efficiency Using the Device

To validate the efficiency of spheroid collection, spheroids cultured on the device were stained with calcium-AM to assess cell viability and enable fluorescence observation. Prior to droplet confrontation, spheroids were presented in droplets on the chip. Following the introduction of liquid from the second injection port, which caused the droplets to burst, the spheroids were allowed to fall in the collection wells of the device.

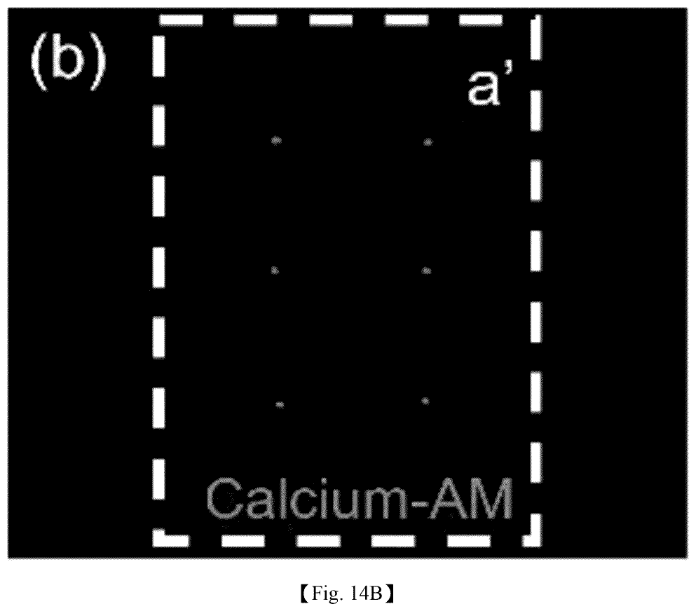

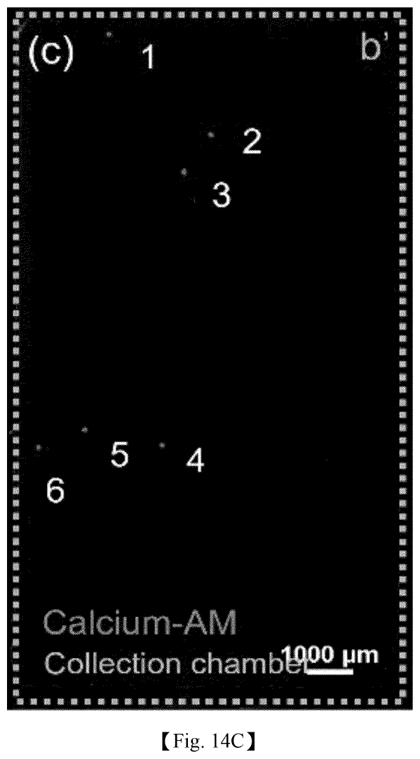

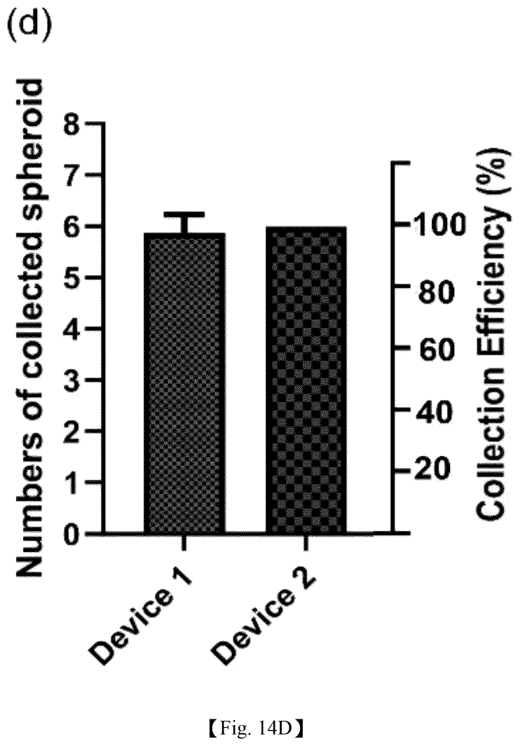

A large scan image (see FIGS. 14A-14D) reveals six individual spheroids that were collected entirely in their respective unit on the μ-collection-slide. The collection efficiency was quantified based on the results shown in FIGS. 14A-14D. The quantitative data indicate that the spheroid collection process is simple and efficient, allowing for robust collection of most spheroids without disrupting the cells or requiring complex manipulation.

From the above description, one skilled in the art can easily ascertain the essential characteristics of the present invention and, without departing from the spirit and scope thereof, can make various changes and modifications of the invention to adapt it to various usages and conditions. Thus, other embodiments are also within the scope of the following claims.

What is claimed is:

1. A cell operation device, comprising:
a plurality of independently operable chip units, each comprising:
a first chamber and a second chamber arranged vertically;
wherein the first chamber is positioned above the second chamber;
wherein the first chamber comprises a microchannel and an array of through-holes disposed on a bottom surface of the microchannel to facilitate droplet formation; and
wherein the second chamber is configured to receive a plurality of spheroids from the droplets and comprises a plurality of collection wells,
wherein each collection well is positioned directly beneath a corresponding droplet formed at the array of through-holes of the first chamber.

2. The device of claim 1, wherein each first chamber further comprises a first injection port configured to receive a sample solution in fluid communication with the microchannel.

3. The device of claim 1, wherein each first chamber further comprises a reservoir in fluid communication with the microchannel for creating hydrostatic pressure.

4. The device of claim 1, wherein the second chamber is disposed on a bottom substrate comprising a transparent glass layer.

5. The device of claim 4, wherein the glass layer has a thickness ranging from approximately 0.1 mm to 0.17 mm.

6. The device of claim 1, wherein the first chamber comprises a second injection port configured to provide fluid communication with the second chamber, adapted to enable infusion of medium into the second chamber until the medium contacts hanging droplets.

7. The device of claim 1, wherein each chip unit further comprises:
a) an inlet formed on the first chamber, configured to introduce medium, and fluidly connected via an internal channel to the second chamber; and
b) an outlet formed on the first chamber, configured to remove medium and fluidly connected via an internal channel to the second chamber.

8. The device of claim 1, wherein the first chamber comprises a plurality of pillars extending toward the second chamber, the pillars being configured to assist in the alignment of the through-holes with the corresponding collection wells in the second chamber.

9. The device of claim 1, wherein the area of the microchannel ranges from 1×1 mm² to 86×128 mm².

10. The device of claim 1, wherein the height of the microchannel ranges from 0.1 mm to 5 mm.

11. The device of claim 1, wherein each through-hole in the array has a diameter ranging from 0.05 mm to 5 mm.

12. The device of claim 1, wherein each through-hole has a depth ranging from 0.1 mm to 5 mm.

13. The device of claim 1, wherein each collection wells are disposed on the transparent glass layer.

14. The device of claim 13, wherein each collection well is shaped as one of the following: rectangular prism, pentagonal prism, heptagonal prism, octagonal prism, triangular prism, square prism, cylindrical prism, or trapezoidal prism.

15. The device of claim 13, wherein each collection well has a planar area ranging from 0.05×0.05 mm² to 86×128 mm².

16. The device of claim 15, wherein each collection well has a height ranging from 0.05 mm to 5 mm.

17. The device of claim 1, wherein the first and second chambers are made of one or more materials selected from the group consisting of polydimethylsiloxane (PDMS), polycarbonate (PC), polymethyl methacrylate (PMMA), polyolefin copolymer (POC), polystyrene (PS), polypropylene (PP), glass, and hydrogel.

18. A method for operating the device of claim 1, comprising:
a) loading a cell suspension into the first chamber via the first injection port;
b) introducing a culture medium into the reservoir of the first chamber to create a hydrostatic pressure difference, thereby forming hanging droplets at the array of through-holes;
c) introducing medium into the second chamber via the second injection port until the medium contacts the hanging droplets.

19. The method of claim 18, further comprising:
d) performing medium replacement by repeatedly removing and adding fresh medium at a volume ranging from 1× to 3×the total channel volume; and

20. The method of claim 19, further comprising:
e) placing the assembled device on a microscope stage to conduct high-resolution confocal imaging of collected spheroids.
